(19) United States
(12) Patent Application Publication
(10) Pub. No.: US 2026/0118352 A1
(43) Pub. Date: Apr. 30, 2026

(54) NANOCOMPOSITES AND METHODS FOR NUCLEIC ACID DETECTION, QUANTITATION AND CHARACTERIZATION

(71) Applicant: iNSAiT Biotronics Incorporated, Taipei City (TW)

(72) Inventors: Wei-Lin Wan, Hsinchu County (TW); Ting-Hao Hsu, Hsinchu County (TW)

(21) Appl. No.: 19/254,037

(22) Filed: Jun. 30, 2025

Related U.S. Application Data
(60) Provisional application No. 63/666,172, filed on Jun. 30, 2024.
(60) Provisional application No. 63/809,474, filed on May 21, 2025.

(51) Int. Cl. G01N 33/543; G01N 21/33; G01N 33/53

(52) U.S. Cl. CPC G01N 33/54346; G01N 21/33; G01N 33/5308

(57) ABSTRACT

The present invention relates to nucleic acid-adsorbing materials and the applications thereof. The nucleic acid-adsorbing materials comprise transition metal nanoparticles coated with one or more modulators, and may further comprise a sensing carrier to form a nanocomposite. The nucleic acid-adsorbing materials can adsorb short or small polynucleotide fragments in a sample solution, which leads to a change in the absorption spectrum and can be detected, quantitated, or characterized with a spectrophotometer.

### RELATED APPLICATION

This application claims the benefit of the U.S. provisional application Nos. 63/666,172 filed on Jun. 30, 2024, and 63/809,474 filed on May 21, 2025. The entirety of the above-mentioned patent applications is incorporated by reference herein and made a part of this specification.

### BACKGROUND OF THE INVENTION

### Field of the Invention

The present invention relates to nucleic acid-adsorbing materials and the applications thereof. In particular, the present invention relates to detecting short/small single strand or double strand nucleic acids with the nucleic acid-adsorbing nanocomposites.

### DESCRIPTION OF RELATED ART

Nucleic acid detection is crucial in many fields such as biotechnology, medicine, agriculture, and environmental science. Common applications include pathogen identification and hereditary diseases diagnosis, and cancer screening. Despite various nucleic acid detection methods already developed, each of them has some drawbacks such as complicated operational procedures, high costs, and/or low sensitivity or specificity. For example, electrophoresis-based techniques have limited quantitative capability in terms of size and concentration. Quantitative polymerase chain reaction (qPCR) requires multiple steps such as reverse transcription, primer design, and target nucleic acid amplification, which makes the method cumbersome and time-consuming. And while Qubit fluorometric quantification can measure concentrations of DNA, RNA, or protein in a sample, this method requires specific instruments with singular functionality, which lead to extra costs. Besides, both qPCR and Qubit utilize fluorophores integrated into double-stranded nucleic acids, and fluorescence signals are generated through binding. Due to the weaker binding stability of fluorophores with small-sized nucleic acids, a fluorescence intensity lower than standard fluorescent signal may result and lead to larger measurement errors for short fragments.

Recently, research of nucleic acids smaller than 100 base pairs or 100 nucleotides, such as microRNA (miRNA) and small interfering RNA (siRNA), has attracted public attention. miRNA has been shown to be crucial indicators in disease studies, diagnosis, therapeutic strategies, and prognosis evaluation. As described above, however, the existing methodology does not provide a convenient, inexpensive approach to detect short or small nucleic acids with good sensitivity.

Accordingly, there is a need to develop a new detection method that offers high sensitivity, high selectivity, and low cost for short and small nucleic acid fragments.

### SUMMARY OF THE INVENTION

The present invention provides nucleic acid-adsorbing materials, and methods for nucleic acid detection, quantitation or characterization using the same. The methods can be performed with a conventional UV/visible spectrophotometer, which detect short/small single stranded or double stranded nucleic acid using label-free nucleic acid-adsorbing materials.

In one aspect, the present invention provides a nucleic acid-adsorbing nanoparticle. The nucleic acid-adsorbing nanoparticle comprises a transition metal nanoparticle and one or more modulators coated on the surface of the transition metal nanoparticle. The transition metal may be gold (Au), silver (Ag), copper (Cu) platinum (Pt), or alloys thereof. In one embodiment, the transition metal nanoparticle is gold nanoparticle. The one or more modulators may be positively charged compounds, such as polyethylenimine (PEI), methylene blue (MB), and poly(diallyl dimethylammonium chloride). The one or more modulators may be carboxyl/hydroxyl-containing compounds, such as citrate. The one or more modulators may be hydrophilic compounds, such as poly(acrylic acid) (PAA). The one or more modulators may be hydrogen bond-forming compounds to such as polyvinylpyrrolidone (PVP), which enables hydrogen bonding with phenolic compounds. The one or more modulators may also be acidic amino acids such as aspartate or glutamate, or may be basic amino acids such as arginine or lysine. The nucleic acid-adsorbing nanoparticle is with a size of nanoscale, especially a size smaller than 100 nm. In one embodiment, the transition metal nanoparticle has an average size of 20 to 40 nm.

In another aspect, the present invention provides a nucleic acid-adsorbing nanocomposite, which comprises a sensing carrier with hydroxyl and carboxyl groups, and a plurality of nucleic acid-adsorbing nanoparticles described above. The plurality of nucleic acid-adsorbing nanoparticles is immobilized on the sensing carrier. In one embodiment, the sensing carrier comprises miniaturized graphene oxide (GO). In another embodiment, the sensing carrier comprises miniaturized reduced graphene oxide (rGO). The sensing carrier may have an O/C_{fit} ratio between 0.4 and 0.8, and may have an average size below 1 μm.

In still another aspect, the present invention provides a method for nucleic acid detection, quantitation or characterization. The method comprises steps of (1) mixing a nucleic acid sample with a nucleic acid-adsorbing material solution to form a testing sample, (2) adding a reaction buffer comprising one or more sensitivity enhancers into the testing sample, (3) measuring absorption of the testing sample in a predetermined wavelength range, and (4) performing an analysis to the measured absorption for nucleic acid detection, quantitation or characterization. In this method, the nucleic acid-adsorbing material comprises a plurality of transition metal nanoparticles, each of which has one or more modulators coated on its surface. In short, the nucleic acid-adsorbing material may be the nucleic acid-adsorbing nanoparticles, or the nucleic acid-adsorbing nanocomposites described above. The sensitivity enhancer may be one or more metal cations, such as lithium ion (Li⁺), sodium ion (Na⁺), potassium ion (K⁺), magnesium ion (Mg²⁺), calcium ion (Ca²⁺), etc. In one embodiment, the sample is let stand for at least 20 minutes before adding the reaction buffer, and further let stand for at least 10 minutes before measuring the absorption profile. The nucleic acid sample may be diluted in a dilution buffer such as TE buffer or DEPC water before mixing with the suspension of nucleic acid-adsorbing material. The absorption of the testing sample can be measured by conventional UV/visible spectrophotometer. For example, the absorption of the testing sample may be measured between wavelengths of 400-800 nm, or between 450-750 nm. The analysis may determine presence of nucleic acid, concentration, proportion of a target size, or single strand/double strand nature of nucleic acid in the testing sample. The target size may be a specific size (e.g., 160 bp, 200 bp, 300 bp, 400 bp, 500 bp or 600 bp) or a size range (e.g., 100-500 bp).

Depending on the modulators and buffer conditions, the nucleic acids suitable for detection, quantitation or characterization with the above method are short or small nucleic acids. In one embodiment, the nucleic acids to be detected are small nucleic acids with 100-500 base pairs in length. In another embodiment, the nucleic acids to be detected are short nucleic acids with less than 50 nucleotides in length. In one embodiment, the modulators comprise citrate, and the nucleic acid-adsorbing material is configured to detect single-stranded nucleic acid which is less than 50 nucleotides in length, such as microRNA (miRNA). In one embodiment, the modulators comprise methylene blue (MB), and the nucleic acid-adsorbing material is configured to detect double-stranded nucleic acid which is less than 50 base pairs in length, such as small interfering RNA (siRNA). In one embodiment, the modulators comprise poly(acrylic acid) (PAA), and the nucleic acid-adsorbing material is configured to detect double-stranded nucleic acid which is between 100 and 500 base pairs in length, such as cell-free DNA (cfDNA).

In yet another aspect, the present invention provides methods of manufacturing a nucleic acid-adsorbing nanoparticle or nucleic acid-adsorbing nanocomposite. In one embodiment, the sensing carrier such as miniaturized rGO is mixed with the transition metal precursor such as HAuCl₄ to form a first mixture to a temperature of 90-100° C. Then the first mixture is reduced with a reducing agent such as sodium citrate and optionally with a protecting agent to form the nanocomposite. In another embodiment, the sensing carrier such as miniaturized rGO is mixed with a solution containing transition metal precursor (e.g. HAuCl₄) and transition metal nanoparticles (e.g. gold nanoparticles) seeds. The mixture is then reduced with a reducing agent (e.g., sodium citrate) at 90-100° C.

Other objectives, advantages and novel features of the invention will become more apparent from the following detailed description when taken in conjunction with the accompanying drawings.

### BRIEF DESCRIPTION OF THE DRAWINGS

FIG. 1 shows the structure of a nucleic acid-adsorbing nanoparticle.

FIG. 2 shows the structure of a nucleic acid-adsorbing nanocomposite.

FIG. 3 shows peak shifts when oligonucleotides or polynucleotides attach to nucleic acid-adsorbing nanocomposites.

FIG. 4 shows nucleic acid-adsorbing nanocomposites with different sizes. The left Eppendorf tube shows nucleic acid-adsorbing nanocomposite prepared by the sensing carrier having a size ≤600 nm, which does not form any aggregates and agglomerates. The right Eppendorf tube shows nucleic acid-adsorbing nanocomposite prepared by the sensing carrier having a size ≥1 μm, which has an elevated amount of precipitation at the bottom.

FIG. 5 shows the size distribution of citrate modified gold nanoparticles (Cit-AuNPs) and PAA modified gold nanoparticles (PAA-AuNPs).

FIG. 6 shows the size distribution of methylene blue modified gold nanoparticles (MB-AuNPs).

FIG. 7 shows the zeta potential distribution of PAA-AuNPs, which has an average zeta potential of −31 mV.

FIG. 8 shows the zeta potential distribution of MB-AuNPs, which has an average zeta potential of 8.35 mV.

FIG. 9A shows absorption spectra for PAA-AuNPs interacting with different kinds of 5 ng/μL double stranded DNA. FIG. 9B shows absorption spectra for different kinds of 10 ng/μL double stranded DNA adsorbed onto PAA-AuNPs.

FIGS. 10A and 10B are absorption spectra for PAA-AuNPs interacting with different kinds of 5 ng/μL double stranded DNA. All spectra in FIG. 10A contain only single DNA species, while three spectra in FIG. 10B are obtained with DNA samples mixing a short fragment (a species shorter than 100 bp) with a small fragment (a species longer than 150 bp).

FIG. 11 shows the absorption profiles of the MB-AuNPs interacting with single stranded DNA (ssDNA), double stranded DNA (dsDNA), and the mixture thereof (i.e. ssDNA/dsDNA).

FIG. 12 is a standard curve for determining the concentration of the dsDNA using MB-AuNPs.

FIG. 13A illustrates the size distribution of two Cit-AuNP-mrGO (citrate modified gold nanoparticles immobilized on miniaturized reduced graphene oxide) samples prepared in the first embodiment. The first sample (No. 1) has an average size of 345.3 nm, and the second sample (No. 2) has an average size of 390.4 nm. FIG. 13B illustrates the absorption spectra of the first sample (No. 1) and the second sample (No. 2) in the region of 500-550 nm. It shows that nanocomposites No. 1 and No. 2 exhibit high absorbances with peak surface plasmon resonance (SPR) at 530 nm and 525 nm, respectively.

FIG. 14A shows the size distribution of Au seeds measured by dynamic light scattering, and FIG. 14B shows the zeta potential of Au seeds measured by zeta potential analysis.

FIGS. 15A and 15B illustrate the zeta potential distribution of two Cit-AuNP-mrGO samples prepared in the first embodiment. The first sample (No. 1), as shown in FIG. 15A, has an average zeta potential of −45.8 mV. The second sample (No. 2), as shown in FIG. 15B, has an average zeta potential of −44.7 mV.

FIGS. 16A and 16B shows the size distribution (FIG. 16A) and the zeta potential distribution (FIG. 16B) of Cit-AuNP-mrGO nanocomposite sample prepared in the second embodiment. The Cit-AuNP-mrGO nanocomposites have a size distribution as shown in FIG. 16A with an average size of approximately 392.7 nm, and a zeta potential distribution as shown in FIG. 16B with an average zeta potential of approximately −44.7 mV.

FIG. 17A shows absorption spectra for No. 1 Cit-AuNP-mrGO nanocomposites of the first embodiment, in which the nanocomposites were interacted with different concentrations (from 0 to 10 ng/μL) of 500 bp double stranded DNA. FIG. 17B shows absorption spectra for No. 2 Cit-AuNP-mrGO nanocomposites of the first embodiment, in which nanocomposites were interacted with different concentrations (from 0 to 10 ng/μL) of 500 bp double stranded DNA.

FIGS. 18A and 18B show the absorption spectra for No. 1 and No. 2 Cit-AuNP-mrGO nanocomposites interacting with DNA samples having different lengths (400, 500 or 600 bp), respectively.

FIGS. 19A and 19B show the absorption profiles of Cit-AuNP-mrGO nanocomposite (the second embodiment) with single stranded DNA (ssDNA), double stranded DNA (dsDNA), and the mixture thereof (i.e. ssDNA/dsDNA).

FIG. 20 is a standard curve for determining the concentration of the ssDNA using Cit-AuNP-mrGO nanocomposites.

### DETAILED DESCRIPTION OF PREFERRED EMBODIMENTS

The terminology used in the description presented below is intended to be interpreted in its broadest reasonable manner, even though it is used in conjunction with a detailed description of certain specific embodiments of the technology. Certain terms may even be emphasized below; however, any terminology intended to be interpreted in any restricted manner will be specifically defined as such in this Detailed Description section.

The present invention provides a nucleic acid-adsorbing nanoparticle, which comprises a metal nanoparticle coated with various modulators. A plurality of nanoparticles may be combined with a sensing carrier to form a nucleic acid-adsorbing nanocomposite. The nucleic acid-adsorbing nanoparticle or the nucleic acid-adsorbing nanocomposite may then be used to detect or quantify or characterize short or small nucleic acids (e.g., oligonucleotides and polynucleotides) in a sample.

### Nucleic Acid-Adsorbing Nanoparticle

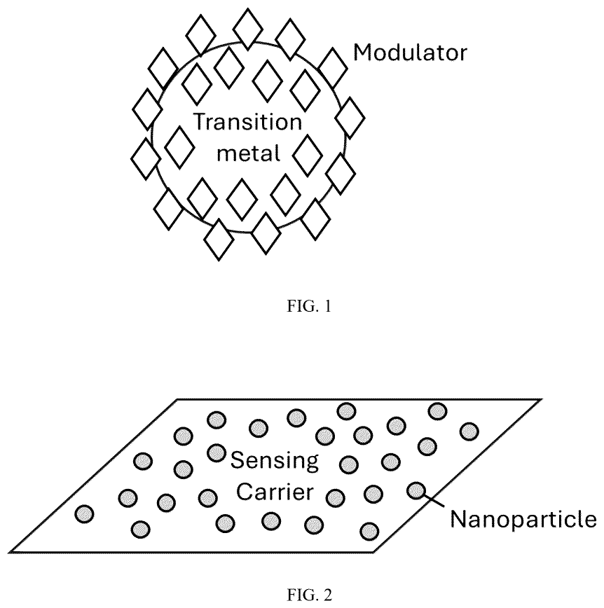

The nucleic acid-adsorbing nanoparticle is a nanoparticle that has affinity with nucleic acid such as DNA and RNA. The nanoparticle may be made of a transition metal nanoparticle modified with one or more chemical modulators, as shown in FIG. 1. The transition metal nanoparticles can be detected by their characteristic signals generated, and the modulators provide moieties that interact with nucleic acids.

The transition metal used in the nanoparticle may be an element in the d-block of the periodic table (groups 3 to 12), such as Au, Ag, Cu, Pt, and alloys thereof. In one embodiment, the transition metal is gold (Au). To provide good nucleic acid detection ability, the transition metal nanoparticle have a size of at most 40 nm. In one embodiment, the average size of transition metal nanoparticle is between 20 to 40 nm.

As described above, the transition metal nanoparticle may be modified with one or more chemical modulators. A modulator is a substance applied to modify the surface properties of the nanoparticles, and thereby enhancing its affinity for specific target molecules. The modulator may be of different kinds, depending on the type of nucleic acid that the nucleic acid-adsorbing nanoparticle designed to attach. For example, the modulator may contain positive charges to provide binding affinity to phosphate groups on nucleic acid. The modulator may contain hydroxyl and/or carboxyl groups to provide binding affinity to nitrogenous bases on nucleic acid. The modulator may contain amine groups to interact with hydrogen bonds on double-stranded nucleic acids. Examples of the modulator include polyethylenimine (PEI), methylene blue, poly(diallyl dimethylammonium chloride), trisodium citrate, aspartate, glutamate, arginine, lysin, polyvinylpyrrolidone, derivatives thereof, etc. In one embodiment, the modulator is citrate. In another embodiment, the modulator is methylene blue. In yet another embodiment, the modulator is poly(acrylic acid) (PAA). A nanoparticle may comprise one or more types of modulators. In some embodiments, different kinds of modulators may be applied to a nanoparticle.

### Nucleic Acid-Adsorbing Nanocomposite

The nanoparticle described above may be used alone, or may be used with other compounds to form a nanocomposite. For example, a plurality of nanoparticles may combine with a sensing carrier to form a nanocomposite. A sensing carrier may serve as a platform or medium for the attachment and immobilization of sensing elements (e.g., the transition metal nanoparticle), which facilitate the recognition and measurement of target molecules (e.g., nucleic acids such as oligonucleotides and polynucleotides), as illustrated in FIG. 2. Combining with a sensing carrier to form a nanocomposite can enhance the sensitivity of nucleic acid detecting with nucleic acid-adsorbing nanoparticles. Suitable choices may include carbon-containing materials containing π-electrons and micropores which provide increased contact area for oligonucleotides and transition metal nanoparticles via π-π interaction, van der Waals force, etc. and enhance the assembly of nucleic acid-adsorbing nanocomposites. In some embodiments, the sensing carrier comprises hydroxyl and carboxyl groups. Examples include but not limited to graphene oxide (GO) and reduced graphene oxide (rGO).

In some embodiments, a sensing carrier such as rGO is further miniaturized to enhance dispersion ability of nanoparticle. The miniaturization process may be performed by a hydrothermal process combining oxidation and ultrasonic vibration, or by a physical homogenization process known in the art. The miniaturized sensing carrier may have an average size below 1 μm. In one embodiment, the miniaturized sensing carrier has an average size in the range of 350-600 nm.

A carbon-containing sensing carrier may be characterized by measuring O/C_{fit} ratio with the formula below:

$$O / {C}_{fit} = \frac{{A}_{C - OH} + {A}_{C = O} + \frac{1}{2} {A}_{C - O - C} + 2 {A}_{O - C = O}}{{A}_{tot}}$$

The O/C_{fit} ratio may be measured by X-ray photoelectron spectroscopy (XPS) analysis. In this formula, A represents the area of the different species, and Aₜₒₜ is the total area of all the C 1s peaks. XPS measures the surface functionalities (C—C, C═C, C—OH, C═O, C—O—C, O—C═O, and π-π*) of a material, and the carbon content (such as C—C and C═C), the oxygen content (such as C—OH, C═O, C—O—C, and O—C═O), and the O/C_{fit} ratio may be calculated accordingly. In some embodiments, the desired O/C_{fit} ratio is in the range of 0.4 to 0.8.

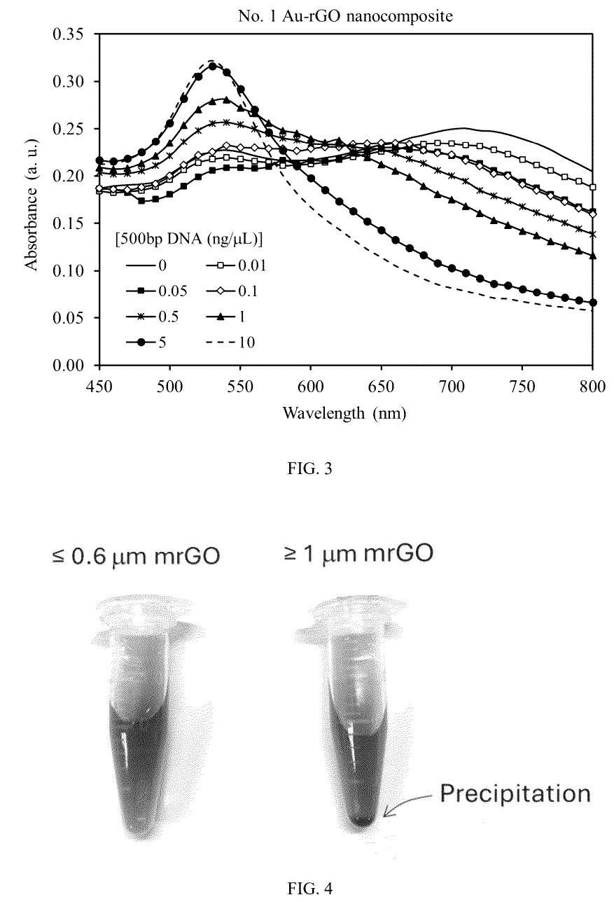

The sensing carrier may be combined with nucleic acid-adsorbing nanoparticles described above to form nucleic acid-adsorbing nanocomposite. In one embodiment, miniaturized rGO is used as the sensing carrier to combine with modulator-modified gold nanoparticles to form a nanocomposite. The nucleic acid-adsorbing nanocomposite is capable of electron transport and produces optical signals in response to incident light. In one example, the nucleic acid-adsorbing nanocomposites exhibit an absorption peak at a wavelength ranging from 500 to 550 nm. When oligonucleotides or polynucleotides attach to the nanocomposites increase, the absorption peak shifts to a shorter wavelength, as illustrated in FIG. 3.

### Preparation of Nucleic Acid-Adsorbing Nanoparticle

The transition metal nanoparticles may be produced by reducing a transition metal precursor with a reducing agent. Optionally, the reducing agent may be used along with a protecting agent to stabilize and protect the transition metal nanoparticles during and after the synthesis of the nucleic acid-adsorbing nanoparticle. The protecting agent prevents agglomeration and maintains the colloidal stability of the transition metal nanoparticles by adsorbing onto their surface, effectively providing a barrier that enhances their dispersion and longevity in solution.

In one example, gold nanoparticle is used as the transition metal nanoparticle, and the examples of the transition metal precursor include HAuCl₄, Au(NO₃)₃, AuBr₃, Au(CH₃COO)₃, K[Au(CN)₂], NaAuCl₄, etc. Examples of the reducing agent include sodium citrate, sodium borohydride, ascorbic acid, oxalic acid, etc. Based on the selected transition metal precursor and reducing agent, the reducing reaction may be performed at room temperature or at a high temperature such as 90-100° C.

### Preparation of Sensing Carrier

As described above, a sensing carrier may be further miniaturized to enhance dispersion ability of nanoparticle. A sensing carrier such as rGO may undergo a hydrothermal process combining oxidation and ultrasonic vibration, or undergo a physical homogenization process known in the art to form a miniaturized sensing carrier such as miniaturized rGO (mrGO). The miniaturization process may be crucial in some embodiments. FIG. 4 shows nucleic acid-adsorbing nanocomposites with sensing carriers having different sizes. The left Eppendorf tube shows nucleic acid-adsorbing nanocomposite prepared by the sensing carrier having a size ≤600 nm, which does not form any aggregates and agglomerates. In contrast, the right Eppendorf tube shows nucleic acid-adsorbing nanocomposite prepared by the sensing carrier having a size ≥1 μm, which has an elevated amount of precipitation at the bottom.

### Preparation of Nucleic Acid-Adsorbing Nanocomposite

There are different approaches to prepare nucleic acid-adsorbing nanocomposites. For example, one approach is to mix the sensing carrier (e.g., mrGO) with the transition metal precursor (e.g. HAuCl₄) before reducing the precursor into nanoparticles. Another approach is to mix the sensing carrier with a solution containing transition metal precursor (e.g. HAuCl₄) and transition metal seeds (e.g., gold nanoparticles), and add a reducing agent to grow the seeds.

In the first approach, the sensing carrier (e.g. mrGO) may be mixed with the transition metal precursor (e.g. HAuCl₄) to form a first mixture to a temperature of 90-100° C. The first mixture is reduced with a reducing agent (e.g., sodium citrate) and optionally with a protecting agent (e.g., sodium citrate).

In the second approach, the sensing carrier (e.g. mrGO) may be mixed with transition metal precursor (e.g. HAuCl₄) and transition metal nanoparticles (e.g. gold nanoparticles) seeds. The mixture is then reduced with a reducing agent (e.g., sodium citrate) at 90-100° C. or reduced with a stronger reducing agent (e.g., sodium borohydride, ascorbic acid, etc.) under room temperature and optionally with a protecting agent (e.g., sodium citrate). The transition metal nanoparticle's seeds are prepared under room temperature using transition metal precursor (e.g. HAuCl₄), stronger reducing agent (e.g., sodium borohydride, ascorbic acid, etc.) and capping agent (e.g., sodium citrate).

### Detection of Nucleic Acid Using Nucleic Acid-Adsorbing Nanocomposites

The nucleic acid-adsorbing nanoparticles or nucleic acid-adsorbing nanocomposites (collectively nucleic acid-adsorbing materials) may be used to detect or quantify nucleic acid such as oligonucleotide or polynucleotide in a sample. The nucleic acid may be deoxyribonucleic acid (DNA) or ribonucleic acid (RNA), and may be single-stranded or double-stranded. The nucleic acid-adsorbing materials disclosed in this invention are especially good at detecting or quantifying short or small nucleic acids in a sample. In one embodiment, the small nucleic acids are DNA or RNA having 100 to 500 base pairs in length. In another embodiment, the short nucleic acids are DNA or RNA having less than 50 nucleotides in length. The nucleic acid to be detected may be single- or double-stranded. Different nucleic acid (e.g., oligonucleotide or polynucleotide) species may be detected with nucleic acid-adsorbing materials modified with different modulators. For example, nucleic acid-adsorbing material modified with citrate may be used to detect single-stranded oligonucleotide which is less than 50 nucleotides in length in the testing sample; the material modified with methylene blue may be used to detect double-stranded polynucleotide which is less than 50 base pairs in length; and the material modified with poly(acrylic acid) (PAA) may be used to detect double-stranded polynucleotide which is between 100 and 500 base pairs in length.

When performing nucleic acid analysis, a nucleic acid sample is mixed with a suspension of a nucleic acid-adsorbing material to form a reaction sample. The reaction sample is placed still for a while (e.g., 10 or 20 min) to enable nucleic acid to be adsorbed onto the nucleic acid-adsorbing material. After that, a reaction buffer comprising a sensitivity enhancer is added into the reaction sample to form a final test sample. Then an absorption of the testing sample at two or more predetermined wavelengths is measured to determine the existence, the quantity, and/or the proportion of a nucleic acid species (e.g., short/small single stranded DNA/RNA).

Sensitivity enhancer is a substance or material containing metal ions (e.g., Li⁺, Na⁺, K⁺, Mg²⁺ or Ca²⁺), which is added to the nucleic acid-adsorbing material to increase its sensitivity towards short oligonucleotides (e.g., ssDNA, ssRNA, dsDNA, or dsRNA) with lengths of 50 nucleotides or less. The sensitivity enhancer functions by altering the electronic or chemical properties of the nucleic acid-adsorbing material, thereby enhancing its interaction with the short oligonucleotides to produce stronger, more precise signals, facilitating accurate detection of these short oligonucleotides.

As described above, the nucleic acid-adsorbing material exhibits an absorption peak in visible light region (e.g., 530 nm). When nucleic acids are adsorbed onto the material, the absorption peak shifts. This shift may be detected and may further be used to characterize the nucleic acid in the sample and quantify the concentration of specific nucleic acid species (e.g., ssDNA). The detection, measurement, and analysis can be obtained using a common (standard) instrument, such as UV/visible spectrophotometers.

For example, when considering the whole spectrum, a nucleic acid-adsorbing material may exhibit different absorption profiles when different types of nucleic acids adsorbed onto the material. A nucleic acid-adsorbing material may also have selectivity to one nucleic acid species over other ones. Therefore, the properties of nucleic acid in the sample such as different lengths and single stand/double strand may be distinguished using suitable nucleic acid-adsorbing material.

When used for quantitation, a responsive curve of a specific nucleic acid species (e.g., short single-stranded polynucleotide) can be plotted by measuring the absorption at a specific wavelength with different nucleic acid concentrations and performing regression analysis to those data points. The regression analysis may comprise methods frequently used in ELISA, which may be a linear regression or non-linear regression method. Specifically, non-linear regression methods such as four-parameter logistic (4PL) and the five-parameter logistic (5PL) regression are often preferred. The concentration of that nucleic acid species in an unknown sample can then be determined by referring to the responsive curve.

Different nucleic acid-adsorbing materials may also be used to determine the purity of specific nucleic acid species in a sample. For example, nucleic acid-adsorbing material modified with citrate may be used to quantitate single-stranded nucleic acid in a sample, and nucleic acid-adsorbing material modified with methylene blue may be used to quantitate double-stranded nucleic acid. When combining these two materials, the purity of single stranded or double stranded species may be determined.

In conclusion, this newly developed method may reduce the cost of purchasing new measurement instruments for nucleic acid analysis.

### EXAMPLES

The following examples are provided to further illustrate the nucleic acid-adsorbing materials and their applications as claimed.

### Example 1: Preparation of Nucleic Acid-Adsorbing Nanoparticles

### Citrate Modified Gold Nanoparticles (Cit-AuNPs)

Gold nanoparticles (AuNPs) were synthesized using a slightly modified Turkevich method. 100 mL solution of 1 mM chloroauric acid (HAuCl₄) was brought to a boil under stirring. Then, 10 mL of 38.8 mM trisodium citrate was quickly added with vigorous stirring and then the solution changed color to ruby red. The solution was further heated for an additional 10-15 minutes. After cooling the solution to room temperature, 3 mL of the synthesized gold nanoparticles solution was transferred to another beaker and mixed with 9 mL of deionized (DI) water.

### Poly(Acrylic Acid) (PAA) Modified Gold Nanoparticles (PAA-AuNPs)

0.48 mL of 0.5 M sodium hydroxide was added into Cit-AuNPs described above to facilitate the ligand exchange and followed by addition of 0.12 mL PAA solution (M.W. 5000, 35%). After sonicated for 1 hour to perform the ligand exchange, the solution was centrifugated at 10,000 rpm for 30 minutes and the supernatant was carefully removed. The PAA-modified gold nanoparticles pellets were re-suspended in DI water.

### Methylene Blue (MB) Modified Gold Nanoparticles (MB-AuNPs)

Gold nanoparticles were synthesized using a slightly modified Turkevich method. 100 mL solution of 1 mM chloroauric acid was brought to a boil under stirring. Then, 10 mL of 38.8 mM trisodium citrate was quickly added with vigorous stirring. The reduction of chloroauric acid by trisodium citrate was practically complete after 10-15 minutes of boiling. After cooling the solution to room temperature, 6 mL of the synthesized gold nanoparticles solution was transferred to another beaker and mixed with 10 mL of deionized (DI) water. After adding 45-50 μL of 1 mM methylene blue, the mixture was rotated for at least 12 hours to complete the modification. After modification, a centrifugation step may be applied to remove excess amount of methylene blue.

### Size Distribution and Zeta Potential Distribution of Nucleic Acid-Adsorbing Nanoparticles

The size distribution and the zeta potential of the nucleic acid-adsorbing nanoparticles are determined by a Dynamic Light Scattering (DLS)/Zeta potential analyzer in an aqueous solution at 25° C.

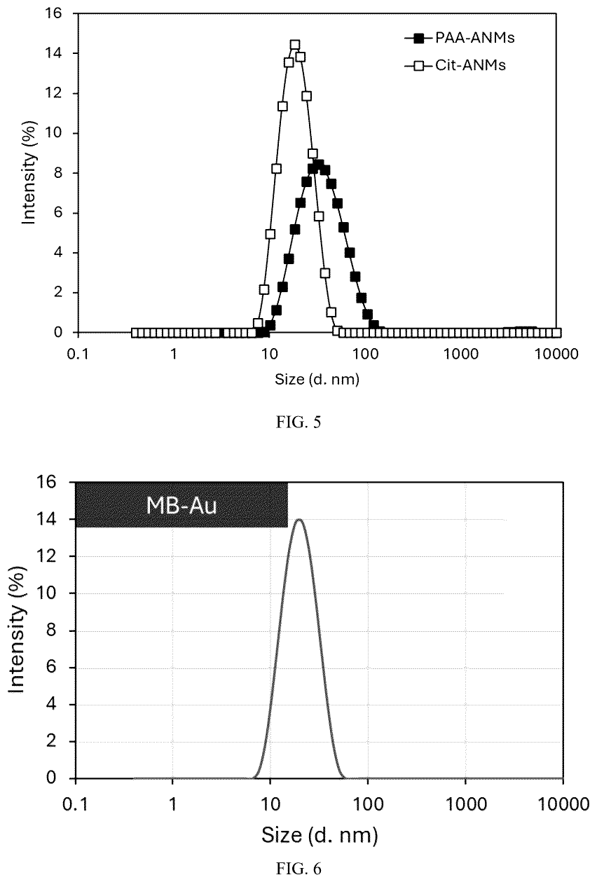

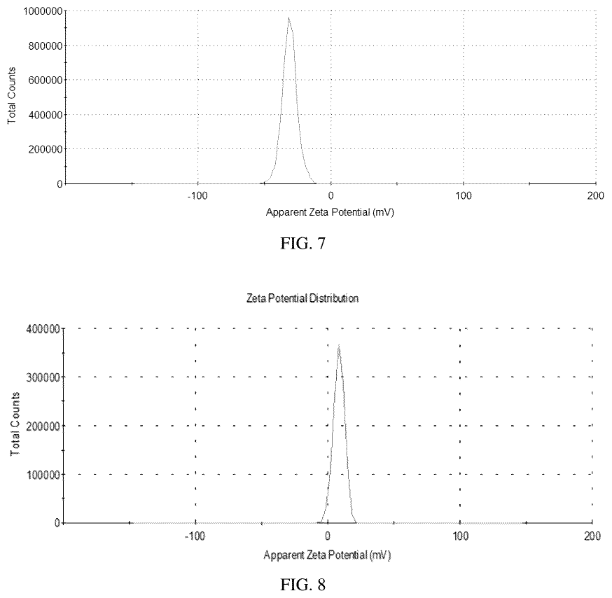

FIG. 5 shows the size distribution of Cit-AuNPs and PAA-AuNPs, and FIG. 6 shows the size distribution of MB-AuNPs. As shown in FIGS. 5-6, Cit-AuNPs, PAA-AuNPs, and MB-AuNPs have an average particle size of 16.73 nm, 38.57 nm and 21.03 nm, respectively. FIG. 7 shows the zeta potential distribution of PAA-AuNPs, and FIG. 8 shows the zeta potential distribution of MB-AuNPs. PAA-AuNPs have an average zeta potential of −31 mV, and MB-AuNPs have an average zeta potential of 8.35 mV.

### Example 2: Nucleic Acid Detection, Quantitation and Characterization Using Nucleic Acid-Adsorbing Nanoparticles

Size Differentiation of dsDNA Using PAA-AuNPs

For the small size DNA fragments (e.g., 100 bp, 300 bp, or 500 bp) measurement, the PAA-AuNP as described in Example 1 was used. The double stranded DNA (dsDNA) samples include 22 bp, 160 bp, 300 bp, 22 bp/160 bp (a sample containing both 22 bp and 160 bp DNA), and 22 bp/300 bp (a sample containing both 22 bp and 300 bp DNA). All the test samples were prepared in 10 μL of sample dilution buffer (50 mM of Tris and 5 mM of MgCl₂). 75 μL of PAA-AuNP solution was added to 10 μL of DNA sample, mixed well, and let stand for at least 20 minutes at room temperature. Then, 15-20 μL of reaction buffer with 1M sodium chloride as sensitivity enhancer was added to each mixture, resulting in a final sodium chloride concentration of 150-200 mM. The sample was further let stand for at least 10 minutes. Finally, the spectra of the reacted PAA-AuNPs were recorded using a SpectraMax® ABS Plus spectrophotometer (an UV/visible spectrophotometer) from 450 nm to 750 nm.

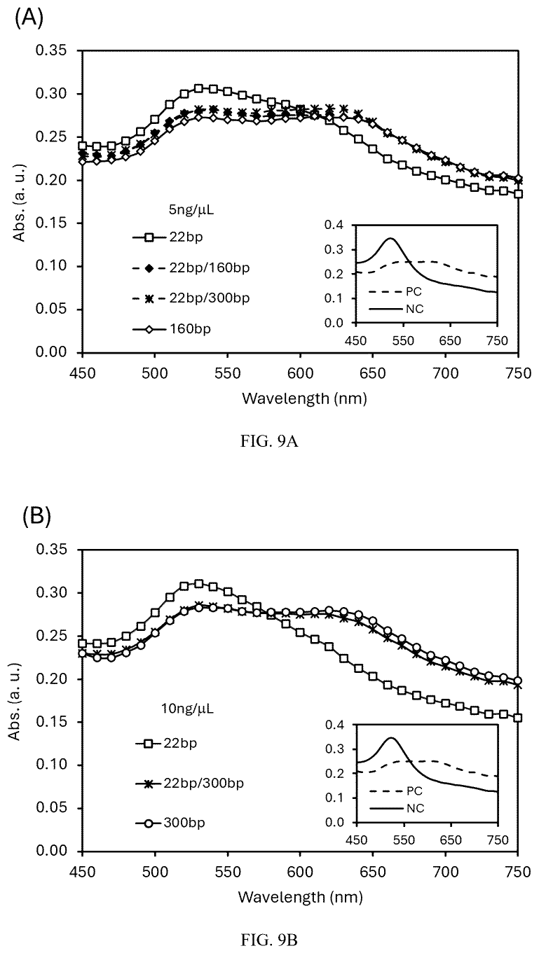

In the 5 ng/μL group as illustrated in FIG. 9A, the testing sample of 22 bp and 160 bp had a DNA concentration of 5 ng/μL, and the testing sample of 22 bp/160 bp and 22 bp/300 bp had a DNA concentration of 5 ng/μL for each DNA species (i.e., 5 ng of 22 bp DNA and 5 ng of 160 bp DNA added in 1 μL dilution buffer for 22 bp/160 bp sample; and 5 ng of 22 bp DNA and 5 ng of 300 bp DNA added in 1 μL dilution buffer for 22 bp/160 bp sample). PC represents “positive control,” which is the sample with PAA-AuNP and sodium chloride but without nucleic acid. NC represents “negative control,” which is the sample with PAA-AuNP only but without nucleic acid and sodium chloride.

In the 10 ng/μL group as illustrated in FIG. 9B, the testing sample of 22 bp and 300 bp had a concentration of 10 ng/μL, and the testing sample of 22 bp/300 bp had a concentration of 10 ng/μL for each DNA species (i.e., 10 ng of 22 bp DNA and 10 ng of 300 bp DNA added in 1 μL dilution buffer). Like FIG. 9A, PC represents “positive control,” and NC represents “negative control.”

The results show that the absorption spectrum of 22 bp DNA fragments is different from that of other longer DNA fragments (e.g., 160 bp and 300 bp). However, the signal of 22 bp would be “masked” if longer DNA fragments are present in the sample.

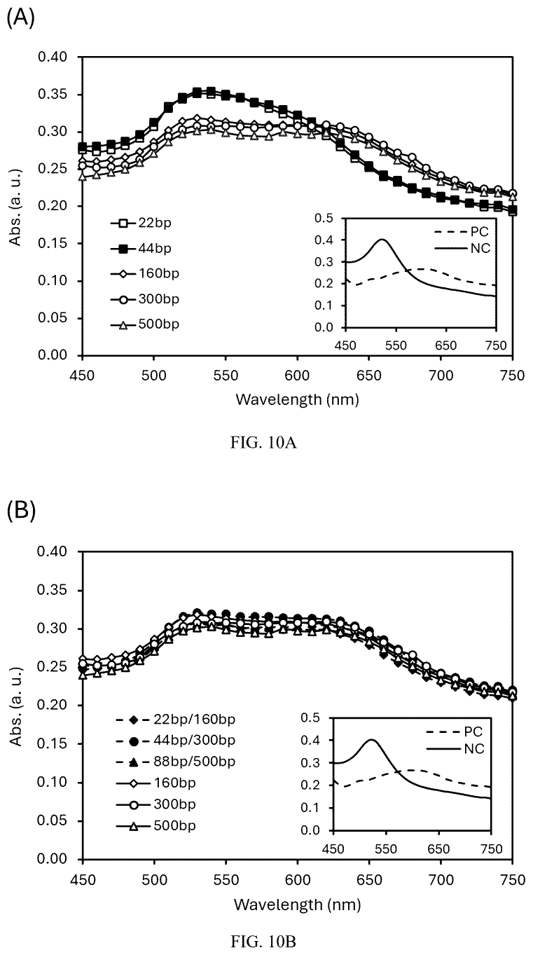

In further testing as shown in FIGS. 10A and 10B, the double stranded DNA (dsDNA) with different sizes of 22 bp, 44 bp, 160 bp, 300 bp, and 500 bp were used. All the test samples were prepared in 10 μL of sample dilution buffer (50 mM of Tris and 5 mM of MgCl₂). 75 μL of PAA-AuNP solution was added to 10 μL of DNA sample, mixed well, and let stand for at least 20 minutes at room temperature. Then, 15-20 μL of 1M sodium chloride was added to each mixture, resulting in a final sodium chloride concentration of 150-200 mM. Each sample was further let stand for at least 10 minutes. Finally, the spectra of the reacted PAA-AuNPs were recorded using a SpectraMax® ABS Plus spectrophotometer from 450 nm to 750 nm.

Each of the testing sample 22 bp, 44 bp, 160 bp, 300 bp and 500 bp had a concentration of 5 ng/μL. The testing sample of 22 bp/160 bp (a sample containing both 22 bp and 160 bp DNA), 44 bp/300 bp (a sample containing both 44 bp and 300 bp DNA) and 88 bp/500 bp (a sample containing both 88 bp and 500 bp DNA) had a concentration of 5 ng/μL for each DNA species (e.g., 5 ng of 22 bp and 5 ng of 160 bp added in 1 μL buffer solution for 22 bp/160 bp sample).

The results show that the absorption spectrum of shorter DNA fragments (e.g., 22 bp and 44 bp DNA fragments) is different from that of longer DNA fragments (e.g., 160 bp and other longer DNA fragments). However, the signal of shorter DNA fragments would be “masked” if longer DNA fragments are present in the sample.

In conclusion, PAA-AuNP can be used to detect presence of small size DNA fragments, such as double stranded DNA ranging from 100 bp to 500 bp. This feature makes it suitable for analyzing cell-free DNA (cfDNA) in a sample.

Detection of ssDNA/dsDNA Using MB-AuNPs

Double stranded nucleic acid such as dsDNA and dsRNA are predominantly hydrophilic due to the present of the phosphate backbone, H-bonds, and phenolic compounds. As described above in Example 1, MB-AuNPs have positive zeta potential. This facilitates the affinity and attachment of dsDNA or dsRNA to the nucleic acid-adsorbing material.

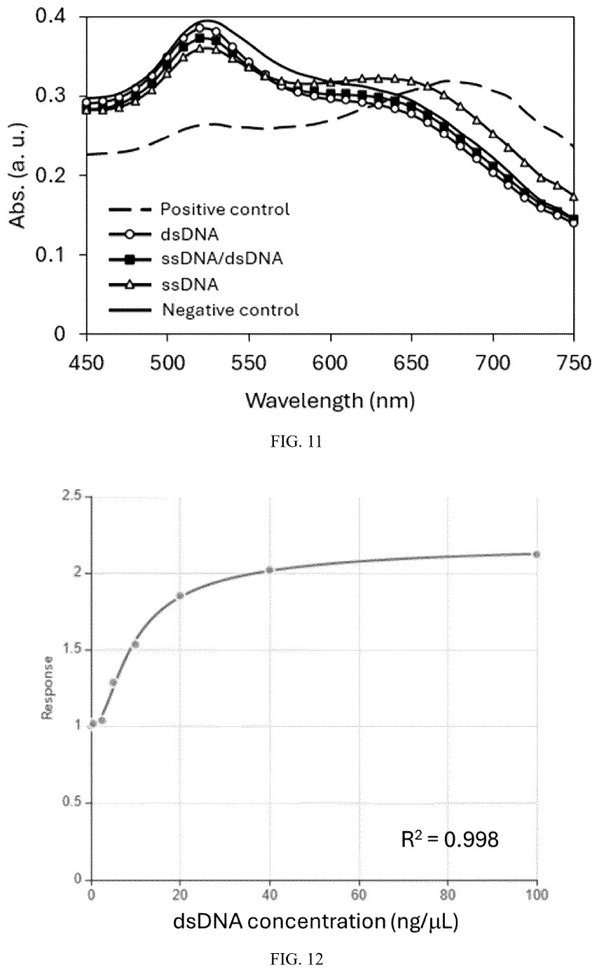

FIG. 11 shows the absorption profiles of the MB-AuNPs interacting with different types of nucleic acids. Single stranded DNA (ssDNA), double stranded DNA (dsDNA), and their combination (i.e. ssDNA/dsDNA) were added to the MB-AuNPs described in Example 1. The dsDNA used herein has less than 50 base pairs and the ssDNA used herein has less than 50 nucleotides.

The prepared samples include 10 ng/μL single stranded DNA (ssDNA), 10 ng/μL double stranded DNA (dsDNA), and a mixture of 10 ng/μL single stranded and 10 ng/μL double stranded DNA (ssDNA/dsDNA). Each nucleic acid sample was prepared in 10 μL of DEPC water. And 90 μL of MB-AuNP solution was added to 10 μL of DNA sample, mixed well, and let stand for at least 20 minutes at room temperature. Then, 2-2.5 μL of reaction buffer with 1M sodium chloride served as a sensitivity enhancer was added to each mixture, resulting in a final sodium chloride concentration of 20-25 mM. Each sample was further let stand for at least 10 minutes. Finally, the spectra of the reacted MB-AuNPs were recorded using a SpectraMax® ABS Plus spectrophotometer from 450 nm to 750 nm. The sample with MB-AuNPs and sodium chloride (but without nucleic acid) is used as a positive control, and the sample with MB-AuNPs only (but without nucleic acid and sensitivity enhancer) is used as negative control.

As shown in FIG. 11, compared with the negative control, the positive control (the sample with MB-AuNPs and sodium chloride) exhibits a higher absorption peak at 650-750 nm. And compared with the positive control, the sample of nucleic acid attached MB-AuNPs remains an absorption peak at 500-550 nm. However, when compared with the ssDNA sample, it shows that the sample of dsDNA attached MB-AuNPs exhibits a higher absorption peak at 500-550 nm under weakly acidic condition (i.e. at around pH 6) in the present of ssDNA with the same DNA concentration. The absorption spectrum of samples containing dsDNA (i.e., dsDNA and ssDNA/dsDNA) is closer to the absorption spectrum of the negative control, while the absorption spectrum of samples containing only ssDNA is closer to the absorption spectrum of the positive control. The increased affinity of MB-AuNPs towards dsDNA can be attributed to the presence of positive charged modulator (i.e. methylene blue) on the surface of the nanoparticles.

MB-AuNPs exhibits substantially identical absorption profiles between dsDNA and ssDNA/dsDNA, which indicates that MB-AuNPs have higher affinity to dsDNA and can be used to determine the concentration and/or proportion of dsDNA. Since RNA molecules are structurally similar to DNA, the selectivity of MB-AuNPs towards short double stranded nucleic acid can also be used to analyze double stranded RNA of similar size, such as small interfering RNA (siRNA).

Quantitation of dsDNA Using MB-AuNPs

FIG. 12 shows a standard curve of the dsDNA for determining the concentration of the dsDNA. To plot this standard curve, samples with different dsDNA concentrations (0, 0.5, 1, 5, 10, 20, 40 and 100 ng/μL) in 10 μL of DEPC water were prepared. 90 μL of MB-AuNP solution was added to 10 μL of dsDNA sample, mixed well and let stand for at least 20 minutes at room temperature. Then, 2-2.5 μL of reaction buffer with 1M sodium chloride served as sensitivity enhancer was added to each mixture, and the sample was further let stand for at least 10 minutes. Finally, the spectra of the reacted MB-AuNPs were recorded using a SpectraMax® ABS Plus spectrophotometer from 450 nm to 750 nm.

Each spectrum is normalized against the positive control (i.e. the sample with MB-AuNPs and sensitivity enhancer). The normalized absorbance ratios of each concentration are obtained by dividing the normalized absorbances at 520 nm by the normalized absorbances at 740 nm. Regression analysis is applied to fit the best curve to the normalized absorbance ratio by the five-parameter logistic (5PL) regression using Microsoft Excel. The R Value (Pearson correlation coefficient) is 0.998, indicating a strong correlation between response values and concentrations. Absorption profiles of two samples with known concentrations (Both concentrations are 10 ng/μL) were used to verify the standard curve. The calculated concentration of the two samples using the measured absorption and the standard curve are 9.01 and 11.24 ng/μL, respectively. The result shows that the error of calculated concentration is less than 15%, indicating a good quantitative ability.

### Example 3: Preparation of Nucleic Acid-Adsorbing Nanocomposites

### Citrate Modified Gold Nanoparticles Immobilized on Miniaturized Reduced Graphene Oxide (Cit-AuNP-mrGO)

In this example, miniaturized reduced graphene oxide (mrGO) is used as the sensing carrier. A commercially available reduced graphene oxide (rGO) solution was first centrifuged at 6,000˜7,000 rpm to remove rGO with larger size. The solution was then subjected to a miniaturization process. The miniaturization process was performed using a homogenizer with high-pressure control. Lastly, 30 g small-size rGO sheets were obtained and modified by mixing 250 mg L-ascorbic acid in 10 mL in the final volume of 100 mL deionized water under a mild condition at 90° C. for 3 hours to obtain miniaturized rGO (mrGO).

In the first embodiment, Cit-AuNP-mrGO nanocomposite is produced by seeded-growth method, wherein gold nanoparticles are used as seeds. Synthesis of Au seeds smaller than 15 nm was done by mixing 5 μmol HAuCl₄ and 5 μmol sodium citrate in a 20 mL aqueous solution to a final concentration of 0.25 mM, respectively. After stirring for 10 min, 0.6 mL NaBH₄ (0.1M) was added to reduce HAuCl₄.

The Cit-AuNP-mrGO nanocomposite was synthesized using modifications of the Turkevich and seeded-growth methods. Briefly, 32.6 μg of synthesized Au seeds and 326 μg of miniaturized rGO (mrGO) were mixed in 34 mL of deionized water, respectively. Then, 1 mL of 10 mM HAuCl₄ was prepared in deionized water and added to the mixture. The whole mixture was heated to a boiling temperature, and then 3 mL of trisodium citrate (38.8 mM) was added. Heating under stirring continued for at least an additional 20˜30 minutes, and the color of the suspension changed to dark red. Then the heating was turned off to let the suspension naturally cool down to room temperature.

In the second embodiment, Cit-AuNP-mrGO nanocomposite is produced by directly reducing a precursor solution (HAuCl₄) in the presence of mrGO. Chloroauric acid (HAuCl₄) was mixed well with mrGO so that the concentrations of chloroauric acid and mrGO are 0.5 mM and 7.5 μg/mL in 20 mL deionized water, respectively. Then 0.4 mL of 194 mM trisodium citrate was quickly added into the mixture with vigorous stirring after boiling. The reduction of chloroauric acid by trisodium citrate was typically complete within 15 minutes of boiling, and the color changed to dark red. The mixture was removed from the hot plate and cooled down to room temperature under stirring, thereby producing Cit-AuNP-mrGO nanocomposites for use with ssDNA/ssRNA.

### Size Distribution and Zeta Potential Distribution of Nucleic Acid-Adsorbing Nanocomposites

Two Cit-AuNP-mrGO nanocomposite samples were prepared in the first embodiment. Characterization of the Cit-AuNP-mrGO nanocomposites was determined using Dynamic Light Scattering (DLS) and referring to the z-average diameter (the intensity-based harmonic mean) and zeta potential, respectively. The absorption spectrum of the nanocomposites was measured using UV/visible spectrophotometers.

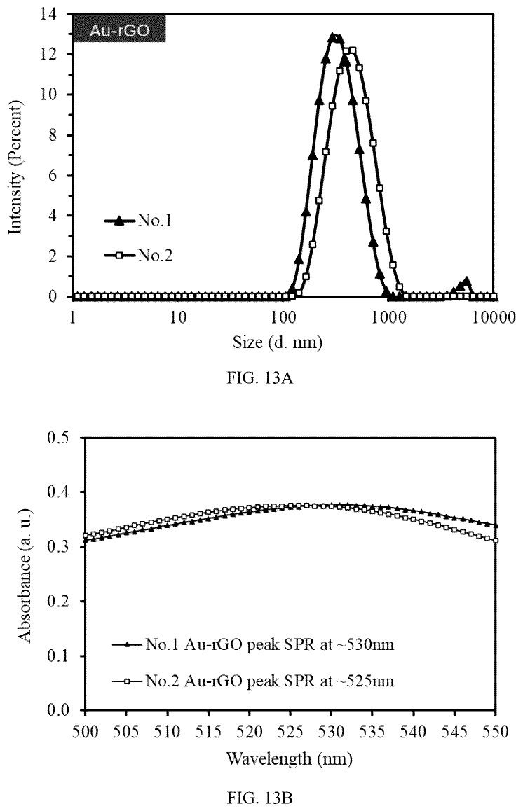

As illustrated in FIG. 13A, the z-average diameters (intensity-based harmonic mean) of nanocomposites No. 1 and 2 are 345.3 nm and 390.4 nm, respectively. FIG. 13B shows that the nanocomposites No. 1 and 2 exhibit high absorption with peak surface plasmon resonance (SPR) at 530 nm and 525 nm, respectively. Based on the absorption spectrum, it is estimated that the size of AuNPs is about 30-40 nm.

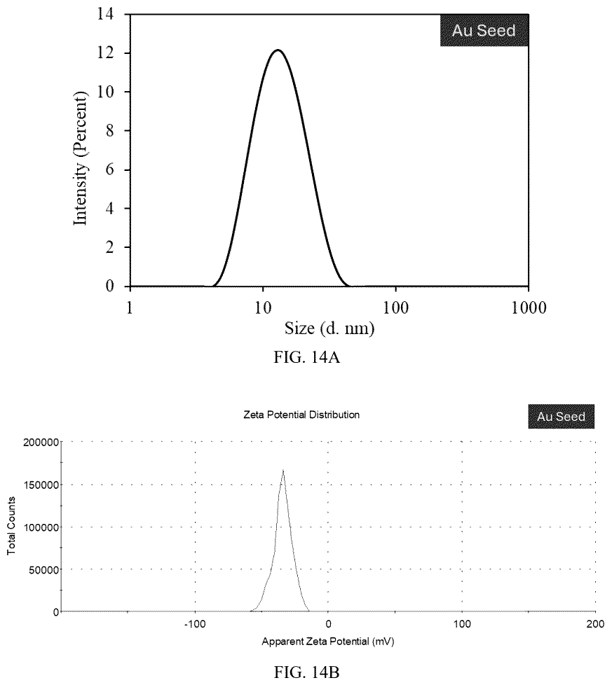

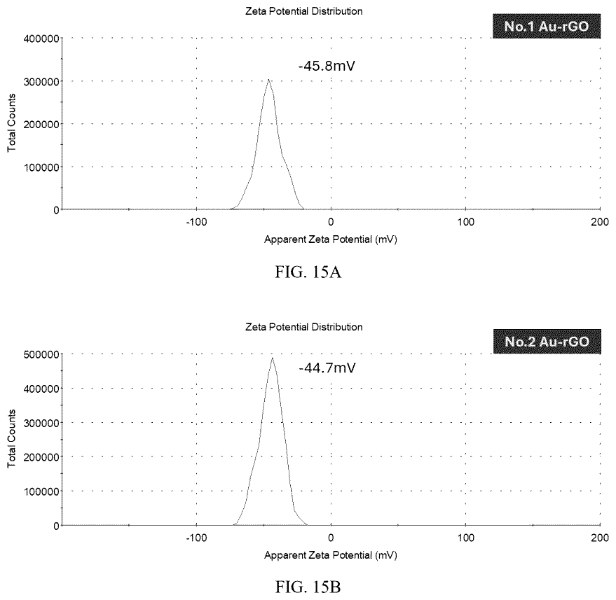

The size distribution of the Au seeds and the zeta potential thereof are as shown in FIGS. 14A and 14B, respectively. And the zeta potential of nanocomposites No. 1 and 2 are as shown in FIGS. 15A and 15B, respectively. It can be seen that the Cit-AuNP-mrGO nanocomposites exhibit strong negative charge compared with Au seeds itself.

The functional groups of Cit-AuNP-mrGO nanocomposites No. 1 and 2 were obtained by X-ray photoelectron spectroscopy (XPS) analysis. The carbon content (C %), oxygen content (0%), and O/C_{fit} ratio were also calculated from the data, as shown in Table 1.

TABLE 1

| Au-rGO | C═C | C—C | C—OH | C═O | O—C═O | C—O—C | π-π* | O/C_{fit} |
|---|---|---|---|---|---|---|---|---|
| No. 1 | 48.15 | 16.75 | 21.21 | 8.65 | 5.23 | 0 | 0 | 0.40 |
| No. 2 | 54.79 | 8.65 | 8.8 | 25.45 | 2.31 | 0 | 0 | 0.38 |

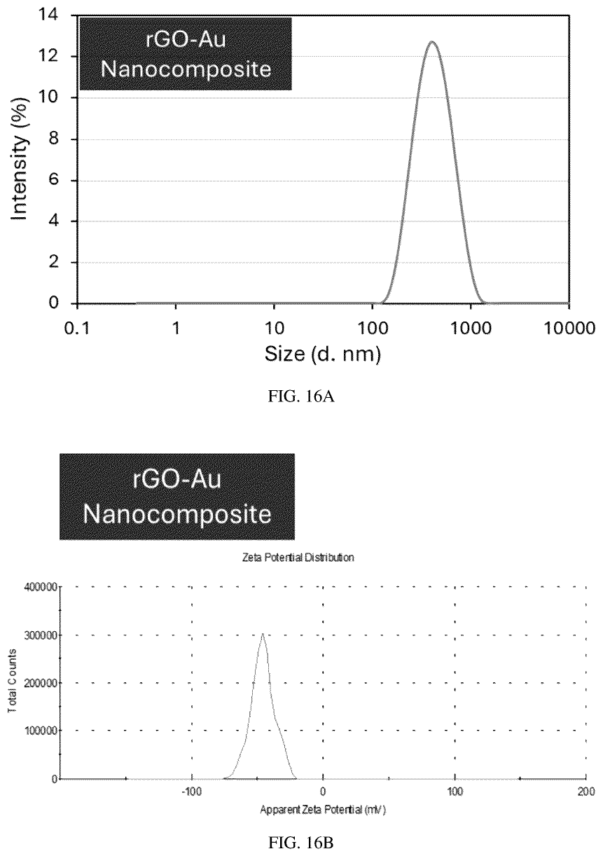

FIGS. 16A and 16B shows the size distribution and the zeta potential distribution of Cit-AuNP-mrGO nanocomposite sample prepared in the second embodiment. The size distribution and the zeta potential of Cit-AuNP-mrGO nanocomposite prepared in the second embodiment are determined by a Dynamic Light Scattering (DLS)/Zeta potential analyzer in an aqueous solution at 25° C. The nanocomposites have an average size of approximately 392.7 nm. The Cit-AuNP-mrGO nanocomposites have an average zeta potential of approximately −44.7 mV. The presence of sodium citrate as protecting agent increases the stability of the nanocomposite.

### Example 4: Nucleic Acid Detection and Quantitation Using Nucleic Acid-Adsorbing Nanocomposites

Concentration and Size Differentiation of dsDNA Using Cit-AuNP-mrGO

Commercially available double stranded DNA (dsDNA) with specific lengths of 400 bp, 500 bp, and 600 bp were prepared and dissolved in 10 μL TE buffer as testing samples.

In the first test, samples with different concentrations (0-10 ng/μL) of 500 bp dsDNA were prepared in 10 μL TE buffer. 80 μL of nanocomposite solution (containing nanocomposite No. 1 or No. 2 produced in the first embodiment) was added to each DNA sample, mixed well and let stand for at least 20 minutes at room temperature. Then, 5-6 μL of 1M sodium chloride was added to each mixture and further let stand for at least 10 minutes. Sodium chloride was added as a sensitivity enhancer to alter the repulsive interactions between nanocomposites that did not interact with nucleic acid. Finally, the spectra of the samples were recorded from 450 nm to 750 nm with SpectraMax® ABS Plus spectrophotometer.

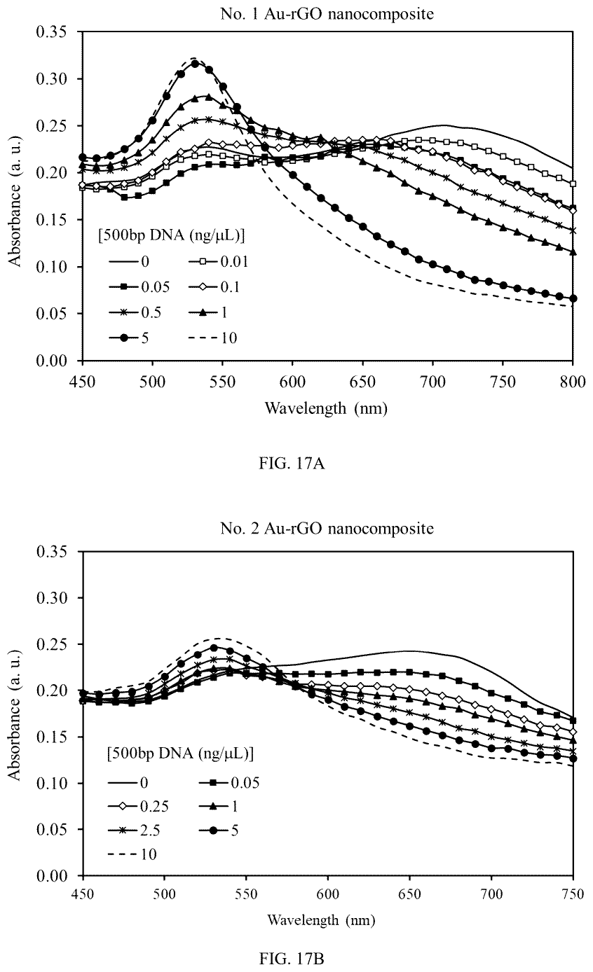

In the first testing as shown in FIGS. 17A and 17B, the absorption spectra of Cit-AuNP-mrGO nanocomposites reacted with different concentrations of 500 bp dsDNA are measured, wherein in FIGS. 17A and 17B the nanocomposites No. 1 and No. 2 are used respectively.

As shown in FIGS. 17A and 17B, the results show an increasing intensity of absorption peaks between 500 nm and 550 nm, accompanied by a decreasing intensity of the absorption peak between 650 nm and 750 nm and between 600 nm and 700 nm as nucleic acid concentration increases, respectively. This phenomenon causes a shift of the absorption peak from 650-750 nm and 600-700 nm to 500-550 nm as nucleic acid concentration increases, respectively. This suggests that the interaction between the nanocomposite and nucleic acids strengthens and stabilizes the nanocomposite against aggregation.

It can be concluded that samples with high DNA concentrations exhibit high absorption, indicating stronger interactions between the nanocomposites and dsDNA than samples with low DNA concentrations. This reduces interference in the reaction at high concentrations, making the nanocomposite suitable for detecting high-concentration nucleic acid samples. Conversely, samples with low DNA concentrations exhibit low absorption and a redshift to 650 nm or higher wavelengths, indicating weaker interactions and greater susceptibility to interference. Samples with different DNA concentrations show different levels of interference result in the absorption spectra, and the distinct signals and reproducible. Consequently, the nanocomposite is suitable for nucleic acid detection and has high sensitivity. It can also be used to determine nucleic acid concentrations.

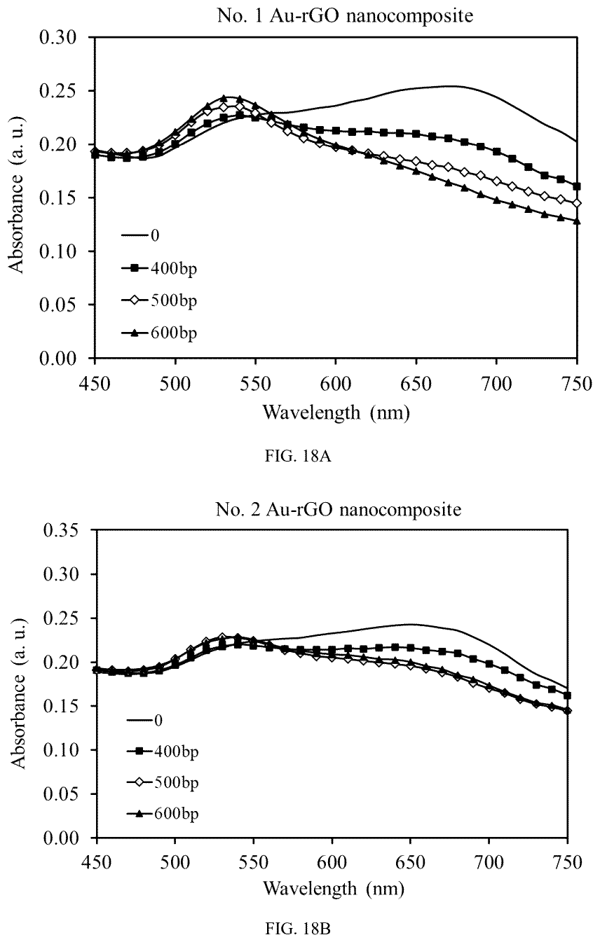

Besides, the Cit-AuNP-mrGO nanocomposite may also be used to analyze size consistency of a target size. FIGS. 18A and 18B show the UV/visible absorption of DNA samples having different lengths (bp). FIGS. 18A and 18B show the results obtained using aqueous solutions containing nanocomposites No. 1 and No. 2, respectively. As shown in FIGS. 18A and 18B, the absorption spectra for 400 bp, 500 bp, and 600 bp DNA samples are distinct from the control (i.e., 0 bp). This indicates that the nanocomposite has different spectral responses in detecting 400 bp, 500 bp, and 600 bp DNA, making it useful for detecting DNA samples' size consistency, and proportion of a target size (e.g., 400 bp, 500 bp, or 600 bp).

Detection of ssDNA/dsDNA Using Cit-AuNP-mrGO

In this example, the absorption profiles of Cit-AuNP-mrGO nanocomposite with different types of nucleic acids were measured. Cit-AuNP-mrGO nanocomposites produced in the second embodiment were used here, and 3 different samples, ssDNA (a sample with 10 ng/μL single stranded DNA), dsDNA (a sample with 10 ng/μL double stranded DNA), and ssDNA/dsDNA (a sample with 10 ng/μL single stranded DNA and 10 ng/μL double stranded DNA), were tested. Each sample was prepared in 10 μL TE buffer. 90 μL of nanocomposite solution was added to 10 μL of DNA sample, mixed well and let stand for at least 20 minutes at room temperature. Then, 4-6 μL of 1M sodium chloride was added to each mixture and further let stand for at least 10 minutes. Finally, the spectra of the reacted nanocomposites from 450 nm to 750 nm were recorded using a SpectraMax® ABS Plus spectrophotometer. The sample with Cit-AuNP-mrGO nanocomposite and sodium chloride (and without any nucleic acid) was used as a positive control, and the sample with only Cit-AuNP-mrGO nanocomposite (but without sensitivity enhancer and nucleic acid) was used as a negative control. The dsDNA used herein was less than 50 base pairs and the ssDNA used herein was less than 50 nucleotides.

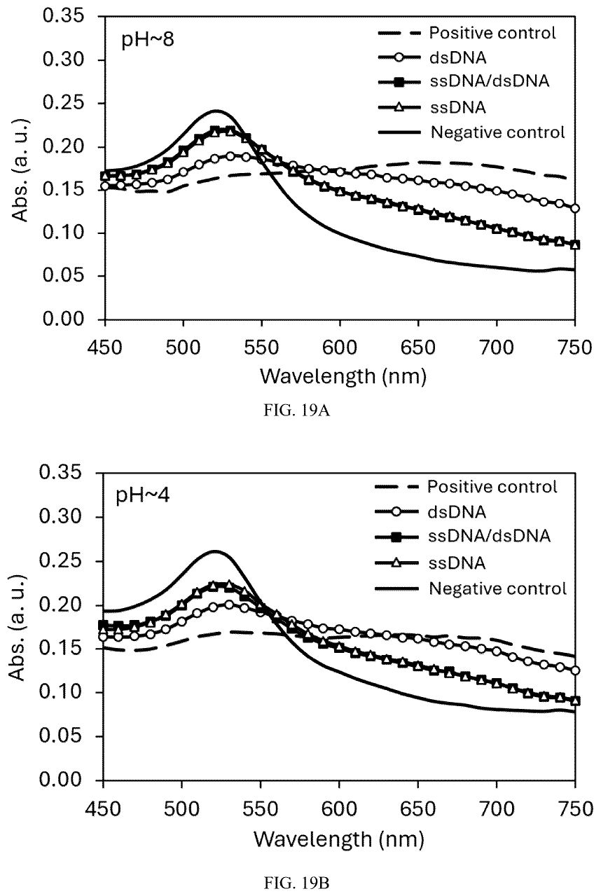

FIGS. 19A and 19B show the absorption profiles of Cit-AuNP-mrGO nanocomposite with different types of nucleic acids. As shown in FIG. 19A, compared with the negative control, the positive control does not exhibit significant absorption peak at 500-550 nm. Compared with the positive control, the attachment of the nucleic acid on the Cit-AuNP-mrGO nanocomposite remains an absorption peak at 500-550 nm. Compared with the dsDNA sample, the Cit-AuNP-mrGO nanocomposite exhibits a higher absorption peak between 500-550 nm under alkaline condition (i.e. at pH 8) in the presence of equal concentration of ssDNA. The absorption spectrum of samples containing ssDNA (i.e., ssDNA and ssDNA/dsDNA) is closer to the absorption spectrum of the negative control, while the absorption spectrum of samples containing only dsDNA is closer to the absorption spectrum of the positive control. The absorption spectra of ssDNA and ssDNA/dsDNA are nearly the same, indicating that the presence of dsDNA does not affect the Cit-AuNP-mrGO nanocomposite for detecting ssDNA. The increased affinity of nucleic acid-adsorbing nanocomposite towards ssDNA can be attributed to the presence of hydroxyl group and the carboxyl group on the surface of the Cit-AuNP-mrGO nanocomposite. Similarly, as shown in FIG. 19B, compared with the dsDNA, the second embodiment of nucleic acid-adsorbing nanocomposite also exhibits a higher absorption peak at a wavelength of 500-550 nm under acidic condition (i.e. at pH 4) in the presence of equal concentration of ssDNA. The Cit-AuNP-mrGO nanocomposite exhibits substantially identical absorption profiles between ssDNA and ssDNA/dsDNA, indicating that the nanocomposite has specificity for ssDNA and can be used for the quantification of ssDNA. It can be concluded that the ability of the Cit-AuNP-mrGO nanocomposite to detect nucleic acids remains unaffected across varying pH levels.

The features described above make Cit-AuNP-mrGO an ideal material to selectively detect and analyze short-sized single stranded nucleic acid. Since RNA molecules are structurally similar to DNA, Cit-AuNP-mrGO can also be used to analyze single stranded RNA of similar size, such as microRNA (miRNA).

Quantitation of ssDNA Using Cit-AuNP-mrGO

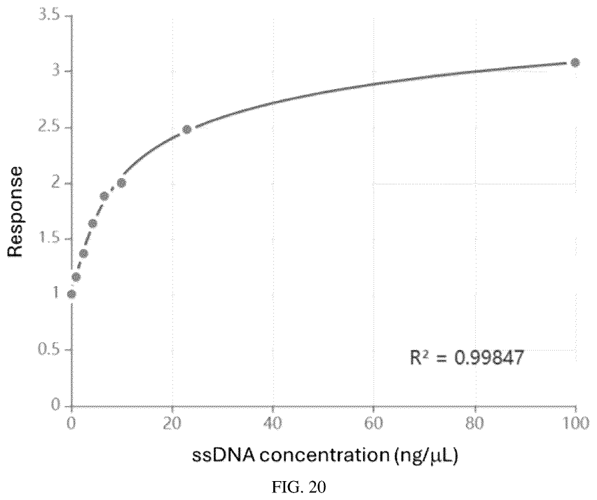

FIG. 20 shows a standard curve of the ssDNA for determining the concentration of the ssDNA. Samples with different ssDNA concentrations (0-100 ng/μL) were used for measurement. In detail, different concentrations of ssDNA dissolved in 10 μL of TE buffer were prepared. 90 μL of Cit-AuNP-mrGO nanocomposite solution was added to 10 μL of ssDNA sample, mixed well and let stand for at least 20 minutes at room temperature. Then, 4-6 μL of 1M sodium chloride served as a sensitivity enhancer was added to each mixture and further let stand for at least 10 minutes.

Finally, the spectra of the reacted nanocomposites from 450 nm to 750 nm were recorded using a SpectraMax® ABS Plus spectrophotometer. The spectra of each sample are normalized to the corresponding positive control (i.e. Cit-AuNP-mrGO nanocomposite and sodium chloride). The normalized absorbance ratios of each concentration are obtained by dividing the normalized absorbances at 520 nm by the normalized absorbances at 710 nm. Five-parameter logistic (5PL) regression is applied to fit the best curve to the normalized absorbance ratio. The R value (Pearson correlation coefficient) is 0.99847, indicating a strong correlation between response values and ssDNA concentrations.

The result shows that Cit-AuNP-mrGO nanocomposite produced in the second embodiment exhibits greater absorption intensity as the ssDNA concentration increases. By plugging the absorbance intensities obtained in FIGS. 19A and 19B in the standard curve, the concentrations of the ssDNA in FIGS. 19A and 19B are 8.955 ng/μL and 11.352 ng/μL, respectively. The error of the calculation results is less than 15%.

Also, when comparing the negative controls in FIG. 11 and FIG. 19 (FIGS. 19A and 19B), the spectrum of MB-AuNPs shows a shoulder peak around 650 nm.

Because Cit-AuNPs have higher affinity towards single stranded nucleic acid and MB-AuNPs have higher affinity towards double stranded nucleic acid, the nucleic acid-adsorbing material containing MB-AuNPs may be combined with the nucleic acid-adsorbing material containing Cit-AuNPs to distinguish the dsDNA from dsDNA/ssDNA and/or vice versa for further purity ratio dsDNA/ssDNA determination.

The foregoing description of embodiments is provided to enable any person skilled in the art to make and use the subject matter. Various modifications to these embodiments will be readily apparent to those skilled in the art, and the novel principles and subject matter disclosed herein may be applied to other embodiments without the use of the innovative faculty. The claimed subject matter set forth in the claims is not intended to be limited to the embodiments shown herein but is to be accorded the widest scope consistent with the principles and novel features disclosed herein. It is contemplated that additional embodiments are within the spirit and true scope of the disclosed subject matter. Thus, it is intended that the present invention covers modifications and variations that come within the scope of the appended claims and their equivalents.

What is claimed is:

1. A nucleic acid-adsorbing nanoparticle, comprising:
a transition metal nanoparticle; and
one or more modulators coated on the surface of the transition metal nanoparticle.

2. The nucleic acid-adsorbing nanoparticle of claim 1, wherein the transition metal nanoparticle is gold nanoparticle having a size of 20 to 40 nm.

3. The nucleic acid-adsorbing nanoparticle of claim 1, wherein the one or more modulators comprise at least one positively charged compound selected from a group consisting of polyethylenimine (PEI), methylene blue (MB), and poly(diallyl dimethylammonium chloride).

4. The nucleic acid-adsorbing nanoparticle of claim 1, wherein the one or more modulators comprise at least one carboxyl/hydroxyl-containing compound.

5. The nucleic acid-adsorbing nanoparticle of claim 4, wherein the at least one carboxyl/hydroxyl-containing compound is citrate.

6. The nucleic acid-adsorbing nanoparticle of claim 1, wherein the one or more modulators comprise poly(acrylic acid) (PAA).

7. A nucleic acid-adsorbing nanocomposite, comprising
a sensing carrier comprising hydroxyl and carboxyl groups; and
a plurality of nucleic acid-adsorbing nanoparticles according to claim 1;
wherein the plurality of nucleic acid-adsorbing nanoparticles is immobilized on the sensing carrier.

8. The nucleic acid-adsorbing nanocomposite of claim 7, wherein the sensing carrier comprises miniaturized graphene oxide (GO) or miniaturized reduced graphene oxide (rGO).

9. The nucleic acid-adsorbing nanocomposite of claim 7, wherein the O/C_{fit} ratio of the sensing carrier is between 0.4 and 0.8.

10. The nucleic acid-adsorbing nanocomposite of claim 7, wherein the nucleic acid-adsorbing nanocomposite has an average size below 1 μm.

11. A method for nucleic acid detection, quantitation and/or characterization, comprising:
mixing a nucleic acid sample with a suspension of a nucleic acid-adsorbing material to form a testing sample;
adding a reaction buffer comprising one or more metal cations into the testing sample;
measuring absorption of the testing sample in a predetermined wavelength range; and
performing an analysis to the measured absorption for nucleic acid detection, quantitation or characterization; wherein:
the nucleic acid-adsorbing material comprises a plurality of transition metal nanoparticles, each of which has one or more modulators coated on its surface.

12. The method of claim 11, wherein the analysis determines presence of nucleic acid, concentration, proportion of a target size, or single strand/double strand nature of nucleic acid in the testing sample.

13. The method of claim 11, wherein the one or more metal cations are selected from a group consisting of lithium ion (Li⁺), sodium ion (Na⁺), potassium ion (K⁺), magnesium ion (Mg²⁺) and calcium ion (Ca²⁺).

14. The method of claim 11, wherein absorption of the testing sample is measured between wavelengths of 400 nm and 800 nm.

15. The method of claim 11, wherein the nucleic acid-adsorbing material further comprises a sensing carrier comprising hydroxyl and carboxyl groups, and wherein the plurality of transition metal nanoparticles is immobilized on the sensing carrier.

16. The method of claim 15, wherein the sensing carrier comprises miniaturized graphene oxide (GO) or miniaturized reduced graphene oxide (rGO).

17. The method of claim 11, wherein the sample is let stand for at least 20 minutes before adding the reaction buffer, and further let stand for at least 10 minutes after adding the reaction buffer.

18. The method of claim 11, wherein the one or more modulators comprise citrate, and the nucleic acid-adsorbing material is configured to detect single-stranded nucleic acid which is less than 50 nucleotides in length in the testing sample.

19. The method of claim 11, wherein the one or more modulators comprise methylene blue, and the nucleic acid-adsorbing material is configured to detect double-stranded nucleic acid which is less than 50 base pairs in length in the testing sample.

20. The method of claim 11, wherein the one or more modulators comprise poly(acrylic acid) (PAA), and the nucleic acid-adsorbing material is configured to detect double-stranded nucleic acid which is between 100 and 500 base pairs in length in the testing sample.
